(19) United States
(12) Plant Patent Application Publication
(10) Pub. No.: US 2006/0075528 P1
(43) Pub. Date: Apr. 6, 2006

(54) PEACH TREE NAMED 'KAY RICH'

(50) PRUNUS PERSICA; PEACH

(76) Inventor: Brian Jones, Reedley, CA (US)

Correspondence Address: MARK D MILLER;KIMBLE, MACMICHAEL & UPTON, 5260 NORTH PALM AVENUE, SUITE 221, FRESNO, CA, 93704, US

(21) Appl. No.: 10/960,476

(22) Filed: Oct. 6, 2004

(51) Int. Cl. A01H 5/00

(52) U.S. Cl. PLT/197

(57) ABSTRACT

A new and distinct variety of peach tree which is somewhat similar to ‘Rich Lady’ (U.S. Plant Pat. No. 7290) peach tree from which it is a bud sport which is distinguished by producing a earlier ripening fruit approximately five days before the fruit produced by the ‘Rich Lady’ peach tree.

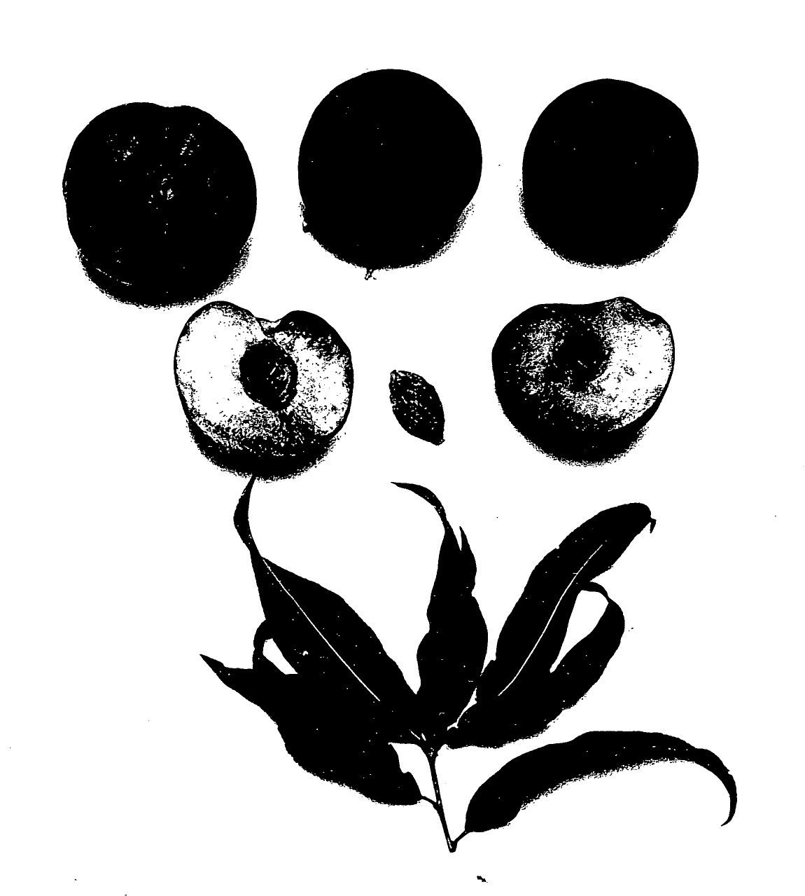

### BACKGROUND OF THE NEW VARIETY

The present invention refers to a new variety of peach which will hereinafter be denominated as ‘KAY RICH’ peach tree which is a semi clingstone fruit which is mature for commercial harvesting and shipment June 4-7 in a normal year in the San Joaquin Valley of Central California as an early market peach with good size and good color.

In the development of new commercial varieties of tree fruits, specific characteristics provide a premium of those varieties which are early or late maturing in the growing season. However, usually early varieties are smaller in size, lack good flavor and/or coloration or a combination of all three characteristics. In some instances that occur with undesirable characteristics, it decreases the possibility of commercial success, e.g. poor storage or holding and shipping capabilities. Therefore, in order for a fruit to be a commercial success it must have a good size, good color, good flavor as well as holding and shipping capabilities. Similarly, the date of maturing must be different from other similar fruit. This new invention meets all of the above aforementioned criteria and thus would be of commercial appeal to the consumer.

### ORIGIN AND ASEXUAL REPRODUCTION OF THE NEW VARIETY

This present invention was discovered by the inventor in a 17-acre peach planting which is located near Kingsburg, Calif. in the San Joaquin Valley of Central California as a bud sport of the ‘Rich Lady’ variety, U.S. Plant Pat. No. 7290, approximately 5-6 years ago. The new invention was grafted on Nemaguard rootstock (2 trees) in 1999/2000 on a nectarine planting north and slightly east of the mother tree site. The asexually reproduced trees first bore fruit in 2003 and 2004. The inventor carefully compared the asexually reproduced trees with the parent sport, including the fruit of the parent, and the progeny are in all respects identical.

### SUMMARY OF THE NEW VARIETY

The ‘KAY-RICH’ peach tree is characterized by producing a large semi-clingstone fruit which has good blush color, flavor and is ripe for commercial harvesting and shipping approximately June 4-7 in the San Joaquin Valley of Central California. The new variety is most similar to ‘Rich Lady’ (U.S. Plant Pat. No. 7290) peach but from which it is distinguishable in that it ripens approximately 5 days before. The fruit of this new variety possesses a very good flavor, good size with a pleasant aroma which makes it greatly acceptable to the consumer.

### BRIEF DESCRIPTION OF THE DRAWING

The accompanying drawing is a color photograph displaying fruit foliage at the bottom of the photograph. Three whole fruit are in the top with the upper left displaying the stem end of the fruit, the upper right displaying the blossom end of the fruit with the fruit in the middle displaying a side view and shape. In the middle a fruit is cut in half with the right side showing the flesh and pit well, a pit in the center with half of the fruit on the right, the flesh and pit in the pit well on the left.

### DETAILED DESCRIPTION

Referring more specifically to the pomological description of this new and distinct variety of peach tree, the following details have been observed under the ecological conditions prevailing in orchard of origin which is located near Kingsburg, Calif. in the San Joaquin Valley of Central California. All major color code designations are by reference to the Dictionary of color by Maerz & Paul, First Edition, 1930. Common color names are occasionally employed.
Tree:
   Size.—medium to large, 12-14 feet in height, 8-10 feet spread.
   Vigor.—early vigor provides a good indicator with over four feet of new growth this growing season.
   Figure (form).—open and spreading with open vase system of training¹
      ¹Four scaffold branches with laterals allowing sunlight for fruit coloration.
   Productivity.—good for fourth leaf tree.
   Regularity of bearing.—normal regular bearing.
   Trunk.—Size — above the graft union 15″ above soil level; circumference is 15″. Color Code — Close of Wineberry (155-E-4) Lenticels — many small Numbers — many Size — small Length: 1-2 mm (0.04-0.079 inch) Color: Close to Pigeon (55-A-4).
   Branches.—Size — medium to large, angle of branches approximately 85°² Circumference — 6 to 7 inches, 28 inches above soil level Surface Texture: Mature — slightly rough Immature — smooth Color (1 year or older) — Shade of Brown (55-A-6 to 55-A-C6) Color (immature) — Grass green (21-L-5) Lenticels — many Size — small, less than 1 mm (less than 0.04 inch).
      ² Ibid.
   Leaves.—Size: Length: 128-143 mm, (5.04-5.94 inch) Width: 30-39 mm (1.18-1.54 inch) Form — lanceolate Color — upper — Brunswick Gr. Lt. (22-L-10) — lower — Fern (21-I-7) Margin Form — Serrate Leaf Vein: Pinnately veined Color — Shadow Green (20-J-8) Thickness — 1 mm (0.04 inches).
   Petioles.—Size: Length: 13-15 mm (0.58-0.59 inch) Width: 1½ mm (0.059 inch) Color — Shadow Green (20-J-3).
   Lenticels.—Number — many, small Size — small, less than 1 mm (less than 0.04 inch) Color — Brown (55-A-6).
   Stemglands.—Size — small: 1-1½ mm (0.04-0.059 inch) Length — less than 0.04 inch Position — two on each side of petiole Color — Mauve Rose (17-E-5) Form — reniform shape.
   Stipule.—None.
Flowers
   Flower buds.—Size: in clusters of 2-3, at pink bud stage Diameter: 7 mm (0.28 inch) average Length: 8-11 mm, average 10 mm (0.39 inch) Shape: Ovate Bud scales: color — Imperial, Cotinga Pr. (45-L-10).
   Flowers.—Diameter: 36-45 mm — avg. 42 mm (1.65 inch) Date of bloom — February 26 — 20% Bloom quality — Abundant Color — Near Vassar Rose (3-I-2) Petals: Size: 5 Color Code: Bottom — Near Vassar Rose (3-I-2) Top — Corinthian Pink (3-C-2) Length: 17-21 mm (avg. 19 mm — 0.75 inch) Width: 18-21 mm (avg. 19 mm — 0.75 inch) Fragrance — Mild Petal claw — very small Length — 2 mm (0.079 inch) Width — 2 mm (0.079 inch) Petal margin — Undulate Pedicel: Length: 4-5 mm (0.16-0.21 inch) Width: 5 mm (0.21 inch) Color — Calliste Green (19-L-6). Calyx: Color Code — Imperial Cotingo Pr. (45-L-10) Surface — Slightly pubescent. Sepals: Size — Length: 10-11 mm (0.39-0.43 inch) Width: 7 mm (0.28 inch) Color Code — Imperial Cotingo Pr (45-L-10). Peduncle: Length: 4-5 mm (0.16-0.21 inch) Width: 5 mm (0.21 inch) Color: Calcite Green (19-L-6). Stamens: Number: 30-35 Length: 8-15 mm — avg. 13 mm (0.51 inch). Filament: Base Color Rose Marie (2-L-5) Upper Color Dark Pink (1-L-5). Anthers: Size: small, less than 1 mm (0.04 inch) Color: New Cocoa, Natal Brown, Mahal+(7-A-10) Pistil: 1 Length: 13-18 mm, average 16 mm (0.63 inch) Color: Viridine Green (17-I-6).
Fruit:
   Size.—Diameter Axial Plane: 58-67 mm (2.28-2.64 inch) Transverse in Suture Plane: 60-76 mm (2.34-2.99 inch) Transverse at Right Angles to Suture Plane: 62-77 mm (2.44-3.03 inch).
   Color.—Blush — Burgundy (56-E-5) Ground Color — Martinique (13-K-11) Flesh — Chipmunk (13-L-9) with flecks or shades of Chianti antique red (6-L-6) Next to Pit, between epidermis and the pit well — Raw Siena (13-L-10).
   Stem cavity.—Length: 30-32 mm (1.18-1.26 inch) Width: 13-15 mm (0.51-0.59 inch) Depth: 12-16 mm (0.047-0.063 inch).
   Stone (pit).—Length: 25-31 mm (0.998-1.22 inch) Width: 21-25 mm (0.84-1.02 inch) Thickness: 17-21 mm (0.67-0.83 inch) Color (dry) — Polignac (4-D-9) Apex — slightly rounded Base — flat Grooves — ad axial side — from shoulder to base — ventral side — from shoulder to base Fibers: many Size: 5-10 mm (0.20-0.34 inch) in length Color: Lt. Chrome Yellow (10-L-4).

The above description of this new variety of peach tree is based on growing conditions near Kingsburg, Calif. in the Central San Joaquin Valley of California. Variations of the usual magnitude and characteristics may occur due to changes in cultural practices including irrigation, fertilization, pruning practices and weather conditions

1. A new distinct variety of peach tree as described and illustrated which is somewhat similar to ‘Rich Lady’ from which it is a bud sport but from which it is distinguished by producing fruits that ripen approximately five days before with good blush color, good size and eating quality which are mature for commercial harvesting and shipping June 4-7, in a normal year.
